(19) United States
(12) Patent Application Publication
(10) Pub. No.: US 2004/0249455 A1
(43) Pub. Date: Dec. 9, 2004

(54) Accommodative intraocular lens system

(76) Inventor: Son Trung Tran, Arlington, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/457,495

(22) Filed: Jun. 9, 2003

(51) Int. Cl. A61F 2/16

(52) U.S. Cl. 623/6.37; 623/6.44; 623/6.4

(57) ABSTRACT

A two-optic accommodative lens system. The first lens has a negative power and is located posteriorly within the capsular bag and lying against the posterior capsule. The periphery of the first optic contains a plurality of generally T-shaped haptics. The overall diameter of the first optic is slightly smaller than the capsular bag. The second optic is located anteriorly to the first optic outside of the capsular bag and is of a positive power. The peripheral edge of the second optic contains a plurality of generally T-shaped haptics and the second optic is slightly larger in overall diameter that the first optic. The haptics allow the second optic to move relative to the first optic along the optical axis of the lens system in reaction to movement of the ciliary muscle and corresponding shrinkage of the capsular bag.

### BACKGROUND OF THE INVENTION

This invention relates generally to the field of intraocular lenses (IOL) and, more particularly, to accommodative IOLs.

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, an opening is made in the anterior capsule and a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

In the natural lens, bifocality of distance and near vision is provided by a mechanism known as accommodation. The natural lens, early in life, is soft and contained within the capsular bag. The bag is suspended from the ciliary muscle by the zonules. Relaxation of the ciliary muscle tightens the zonules, and stretches the capsular bag. As a result, the natural lens tends to flatten. Tightening of the ciliary muscle relaxes the tension on the zonules, allowing the capsular bag and the natural lens to assume a more rounded shape. In the way, the natural lens can be focus alternatively on near and far objects.

As the lens ages, it becomes harder and is less able to change shape in reaction to the tightening of the ciliary muscle. This makes it harder for the lens to focus on near objects, a medical condition known as presbyopia. Presbyopia affects nearly all adults over the age of 45 or 50.

Prior to the present invention, when a cataract or other disease required the removal of the natural lens and replacement with an artificial IOL, the IOL was a monofocal lens, requiring that the patient use a pair of spectacles or contact lenses for near vision. Advanced Medical Optics has been selling a bifocal IOL, the Array lens, for several years, but due to quality of issues, this lens has not been widely accepted.

Several designs for accommodative IOLs are being studied. For example, several designs manufactured by C&C Vision are currently undergoing clinical trials. See U.S. Pat. Nos. 6,197,059, 5,674,282, 5,496,366 and 5,476,514 (Cumming), the entire contents of which being incorporated herein by reference. The lens described in these patents is a single optic lens having flexible haptics that allows the optic to move forward and backward in reaction to movement of the ciliary muscle. A similar designs are described in U.S. Pat. No. 6,302,911 B1 (Hanna), U.S. Pat. Nos. 6,261,321 B1 and 6,241,777 B1 (both to Kellan), the entire contents of which being incorporated herein by reference. The amount of movement of the optic in these single-lens systems, however, may be insufficient to allow for a useful range of accommodation. In addition, as described in U.S. Pat. Nos. 6,197,059, 5,674,282, 5,496,366 and 5,476,514, the eye must be paralyzed for one to two weeks in order for capsular fibrosis to entrap the lens that thereby provide for a rigid association between the lens and the capsular bag. In addition, the commercial models of these lenses are made from a hydrogel or silicone material. Such materials are not inherently resistive to the formation of posterior capsule opacification (“PCO”). The only treatment for PCO is a capsulotomy using a Nd:YAG laser that vaporizes a portion of the posterior capsule. Such destruction of the posterior capsule may destroy the mechanism of accommodation of these lenses.

There have been some attempts to make a two-optic accommodative lens system. For example, U.S. Pat. No. 5,275,623 (Sarfarazi), WIPO Publication No. 00/66037 (Glick, et al.) and WO 01/34067 A1 (Bandhauer, et al), the entire contents of which being incorporated herein by reference, all disclose a two-optic lens system with one optic having a positive power and the other optic having a negative power. The optics are connected by a hinge mechanism that reacts to movement of the ciliary muscle to move the optics closer together or further apart, thereby providing accommodation. In order to provide this “zoom lens” effect, movement of the ciliary muscle must be adequately transmitted to the lens system through the capsular bag, and none of these references disclose a mechanism for ensuring that there is a tight connection between the capsular bag and the lens system. In addition, none of these lenses designs have addressed the problem with PCO noted above.

Therefore, a need continues to exist for a safe and stable accommodative intraocular lens system that provides accommodation over a broad and useful range.

### BRIEF SUMMARY OF THE INVENTION

The present invention improves upon the prior art by providing a two-optic accommodative lens system. The first lens has a negative power and is located posteriorly within the capsular bag and lying against the posterior capsule. The periphery of the first lens contains a plurality of generally T-shaped haptics. The overall diameter of the first lens is slightly smaller than the capsular bag. The second lens is located anteriorly to the first lens outside of the capsular bag and is of a positive power. The peripheral edge of the second lens contains a plurality of generally T-shaped haptics and the second lens is slightly larger in overall diameter that the first lens. The haptics allow the second lens to move relative to the first lens along the optical axis of the lens system in reaction to movement of the ciliary muscle and corresponding shrinkage of the capsular bag.

Accordingly, one objective of the present invention is to provide a safe and biocompatible intraocular lens.

Another objective of the present invention is to provide a safe and biocompatible intraocular lens that is easily implanted in the posterior chamber.

Still another objective of the present invention is to provide a safe and biocompatible intraocular lens that is stable in the posterior chamber.

Still another objective of the present invention is to provide a safe and biocompatible accommodative lens system.

These and other advantages and objectives of the present invention will become apparent from the detailed description and claims that follow.

### BRIEF DESCRIPTION OF THE DRAWINGS

FIG. 1 is an enlarged top plan view of the first embodiment of the second lens of the lens system of the present invention.

FIG. 2 is an enlarged cross-sectional view of the first embodiment of the second lens of the lens system of the present invention taken at line 2-2 in FIG. 1

FIG. 3 is an enlarged top plan view of the first lens of the lens system of the present invention.

FIG. 4 is an enlarged cross-sectional view of the first lens of the lens system of the present invention taken at line 4-4 in FIG. 3.

FIG. 5 is an enlarged top plan view of the first embodiment of the lens system of the present invention.

FIG. 6 is an enlarged cross-sectional view of the first embodiment of the lens system of the present invention taken at line 6-6 in FIG. 5.

FIG. 7 is an enlarged perspective view of the first embodiment of the lens system of the present invention.

FIG. 8 is an enlarged top plan view of the first embodiment of the lens system of the present invention similar to FIG. 5, but showing an alternative orientation of the first lens to the second lens.

FIG. 9 is an enlarged cross-sectional view of the first embodiment of the lens system of the present invention taken at line 9-9 in FIG. 8 similar to FIG. 6, but showing an alternative orientation of the first lens to the second lens.

FIG. 10 is an enlarged perspective view of the first embodiment of the lens system of the present invention similar to FIG. 7, but showing an alternative orientation of the first lens to the second lens.

FIG. 11 is an enlarged top plan view of the second embodiment of the second lens of the lens system of the present invention.

### DETAILED DESCRIPTION OF THE INVENTION

As best seen in the figures, lens system 10 of the present invention generally consists of posterior lens 12 and anterior lens 14. Lens 12 is preferably formed with an overall diameter or length slightly less than the natural lens capsule, for example, around 9.5 millimeters and lens 14 is preferably formed with an overall diameter or length slightly more than the natural lens capsule, for example, around 10.5 millimeters. Lens 12 preferably is made from a soft, foldable material that is inherently resistive to the formation of PCO, such as a soft acrylic. Lens 14 preferable is made from a soft, foldable material such as a hydrogel, silicone or soft acrylic. Lens 12 may be any suitable power, but preferably has a negative power. Lens 14 may also be any suitable power but preferably has a positive power. The relative powers of lenses 12 and 14 should be such that the axial movement of lens 14 toward or away from lens 12 should be sufficient to adjust the overall power of lens system 10 at least one diopter and preferably, at least three to four diopters, calculation of such powers of lenses 12 and 14 being within the capabilities of one skilled in the art of designing ophthalmic lenses by, for example, using the following equations:

P=P₁+P₂−T/n*P₁P₂ (1)

δP=−δT/n*P₁P₂ (2)

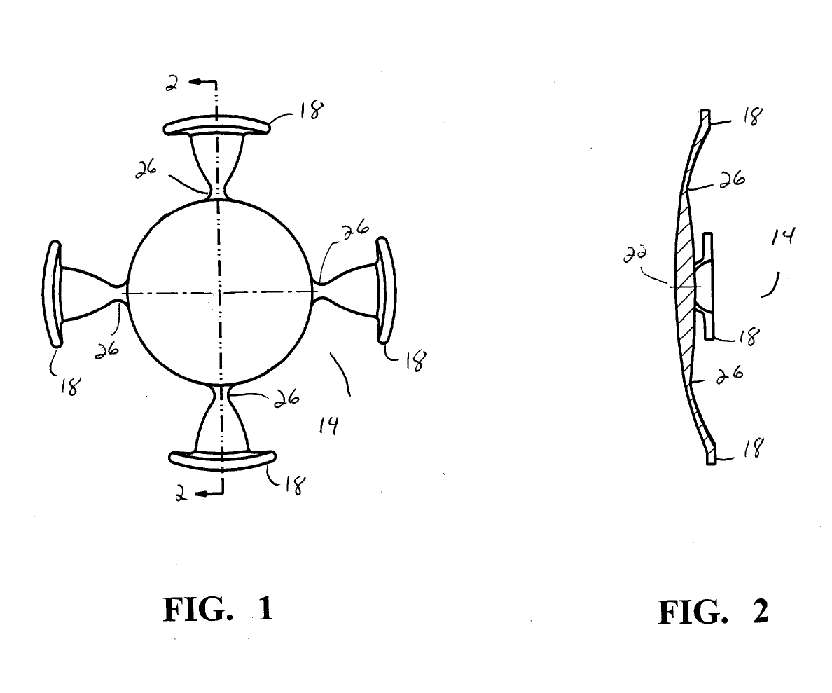

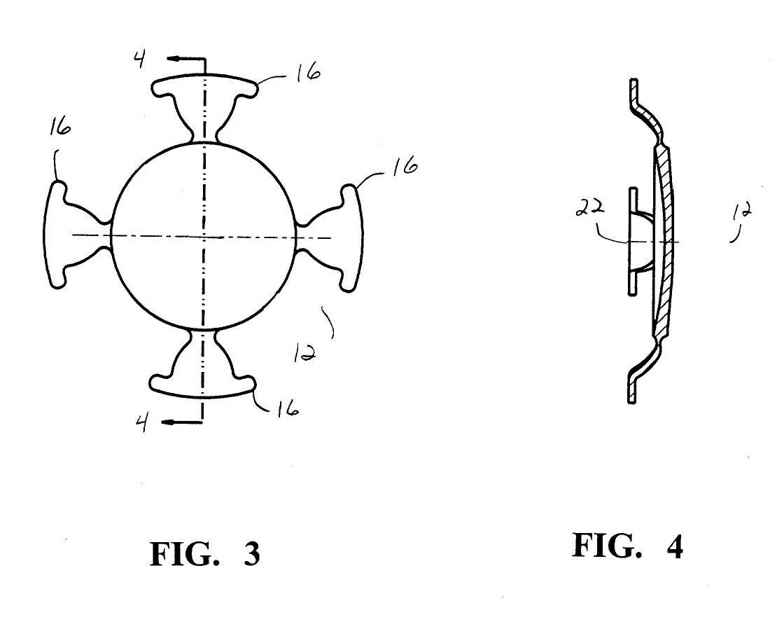

As best seen in FIGS. 1 and 2, lens 12 is generally symmetrical about optical axis 22 and contains a plurality of opposing, generally T-shaped haptics 16 that are shaped to not quite fill the equatorial region of the capsular bag when the zonules are contracted and the capsular bag is at its largest diameter. Haptics 16 are relatively stiff, so as to allow some, but not excessive, flexing in response to ciliary muscle contraction and relaxation. As best seen in FIG. 1, lens 14 contains a plurality of opposing, generally T-shaped haptics 18 that are connected to lens 14 by relatively flexible hinge regions 26 that allow lens 14 to flex more than lens 12 in response to ciliary muscle contraction and relaxation.

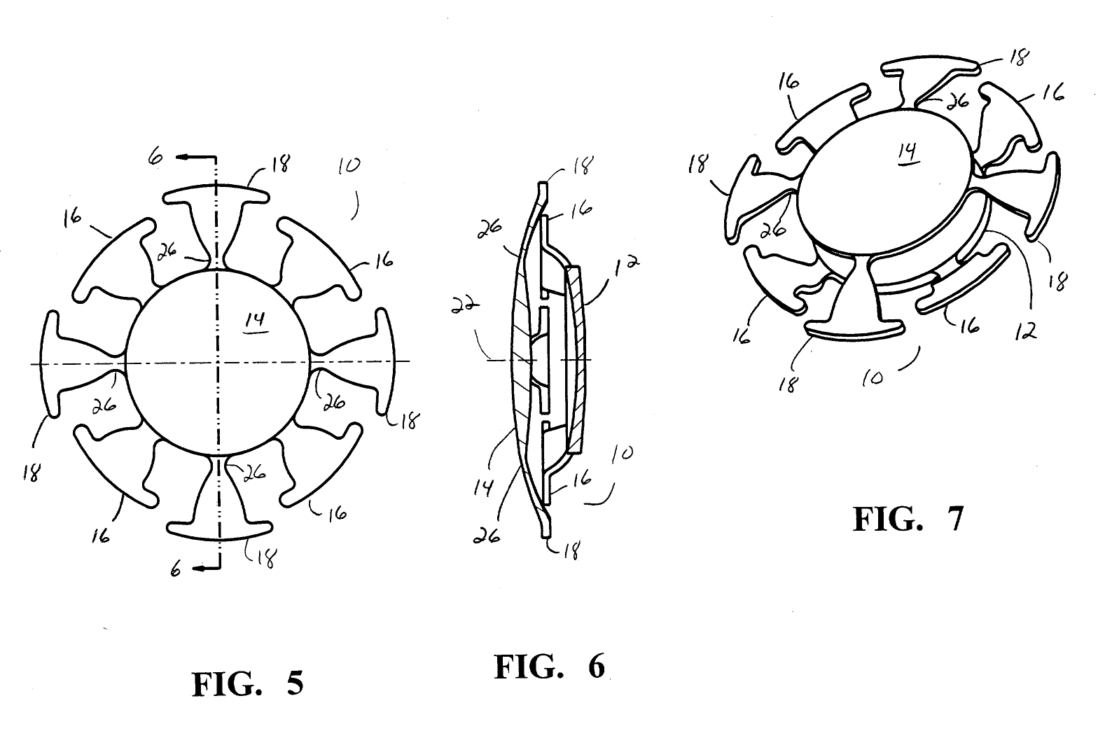

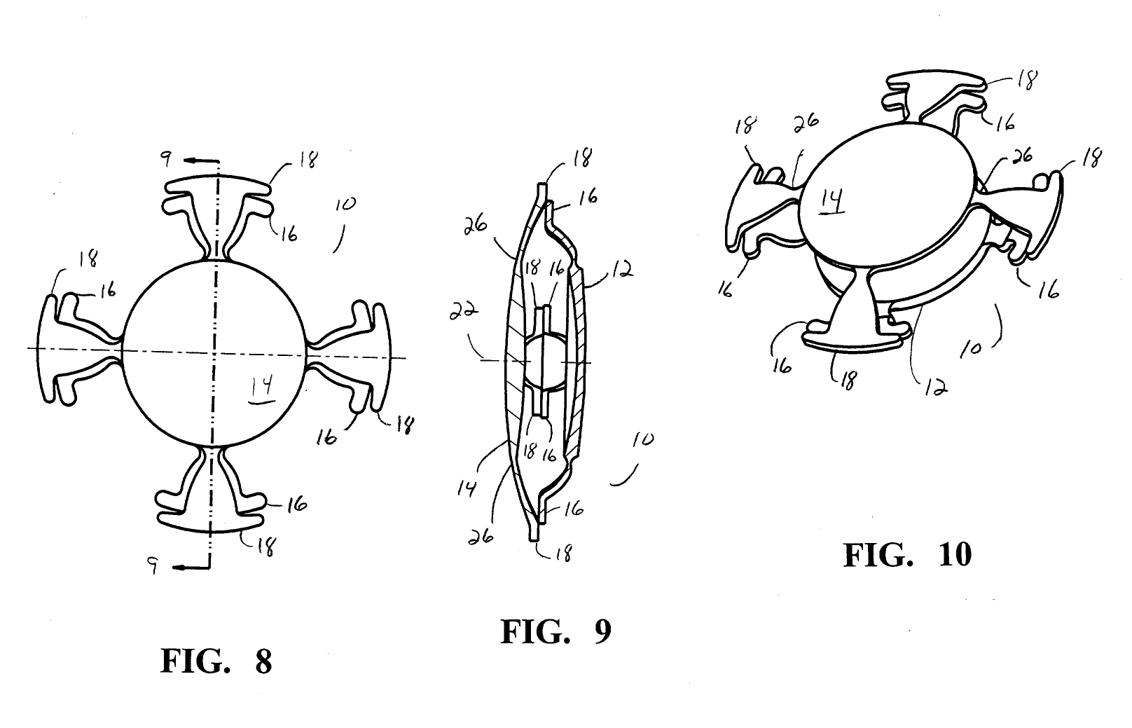

As best seen in FIGS. 5-10, lens 12 is implanted into the capsular bag prior to the implantation of lens 14. Lens 12 is held within the capsular bag by haptics 16 and the properties of the material used to make lens 12. Lens 14 is implanted so that either haptics 16 and 18 are interspersed, as shown in FIGS. 5-7, or overlay each other, as shown in FIG. 8-10, and lenses 12 and 14 are free-floating and not connected to each other. Upon implantation of lens 14, lens 14 will be flexed at hinge regions 26 and vault anteriorly along optical axis 22 because of the slightly larger overall diameter of lens 14 relative to the capsular bag. During contraction and relaxation of the ciliary muscles, both lens, 12 and lens 14 will move, but lens 14 will move a greater amount relative to 12 along optical axis 22 because of hinge regions 26.

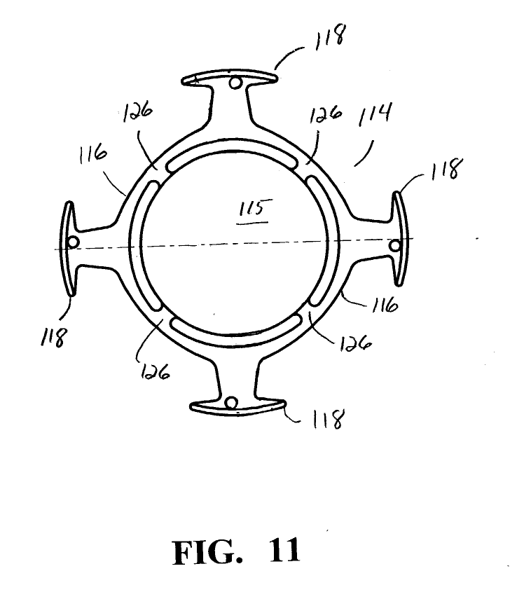

Alternatively, as best seen in FIG. 11, lens 114 may contain optic 115 attached to peripheral ring 116 by hinge regions 126. Ring 116 contains a plurality of opposing, generally T-shaped haptics 118. Lens 114 is preferably formed with an overall diameter or length slightly more than the natural lens capsule, for example, around 10.25-10.5 millimeters. During contraction of the ciliary muscles as described above, this constriction is transferred to band ring 116 through haptics 118 thereby flexing hinges 126. The flexing of hinges 126 causes optic 115 to be pushed anteriorly along visual axis 22.

This description is given for purposes of illustration and explanation. It will be apparent to those skilled in the relevant art that changes and modifications may be made to the invention described above without departing from its scope or spirit.

1. an intraocular lens system, comprising:
a) a first lens having a first plurality of generally t-shaped haptics, the first lens having a first diameter; and
b) a second lens having a second diameter, the second diameter being larger than the first diameter, the second lens including a second plurality of generally t-shaped haptics attached to the second lens by hinge regions,
wherein the first lens and the second lens are free-floating relative to each other.

2. The lens system of claim 1 wherein the hinge regions allow the second lens to vault away from the first lens in reaction to compression of the second lens.

3. The lens system of claim 1 wherein the first lens and the second lens comprise a soft acrylic material.

4. The lens system of claim 1 wherein the second lens comprises a hydrogel material.

5. The lens system of claim 1 wherein the second lens comprises a silicone material.

6. The lens system of claim 1 wherein the hinge regions allow the second lens to move along an optical axis an amount greater than any movement of the first lens along the optical axis in reaction to compression of the first and second lenses.

7. An intraocular lens system, comprising:
a) a first lens having a first plurality of generally t-shaped haptics, the first lens having a first diameter;
b) a second lens having a second diameter, the second diameter being larger than the first diameter; and
c) a peripheral ring surrounding the second lens, the peripheral ring including a second plurality of generally t-shaped haptics, the peripheral ring being attached to the second lens by hinge regions,
wherein the first lens and the second lens are free-floating relative to each other.

8. The lens system of claim 7 wherein the hinge regions allow the second lens to vault away from the first lens in reaction to compression of the second lens.

9. The lens system of claim 7 wherein the first lens and the second lens comprise a soft acrylic material.

10. The lens system of claim 7 wherein the second lens comprises a hydrogel material.

11. The lens system of claim 7 wherein the second lens comprises a silicone material.

12. The lens system of claim 7 wherein the hinge regions allow the second lens to move along an optical axis an amount greater than any movement of the first lens along the optical axis in reaction to compression of the first and second lenses.
